(19) United States
(12) Patent Application Publication
(10) Pub. No.: US 2005/0230423 A1
(43) Pub. Date: Oct. 20, 2005

(54) APPLICATORS FOR LIQUID HOT MELT ADHESIVE AND METHODS OF APPLYING LIQUID HOT MELT ADHESIVE

(76) Inventors: John M. Riney, Buford, GA (US); Duane J. Fisher, Cumming, GA (US); Raymond J. Slattery III, Akron, OH (US)

Correspondence Address: WOOD, HERRON & EVANS, LLP (NORDSON), 2700 CAREW TOWER, 441 VINE STREET, CINCINNATI, OH, 45202, US

(21) Appl. No.: 10/907,136

(22) Filed: Mar. 22, 2005

Related U.S. Application Data
(60) Provisional application No. 60/562,043, filed on Apr. 14, 2004.

(51) Int. Cl. B67D 5/62; A61F 13/15

(52) U.S. Cl. 222/146.2; 156/578

(57) ABSTRACT

Applicators and dispensing systems for dispensing liquid hot melt adhesives and methods of dispensing liquid hot melt adhesives. The dispensing system supplies the liquid hot melt adhesive in a non-activated state to the applicator. The liquid hot melt adhesive is processed to modify at least one activation-sensitive property before dispensing onto a substrate. The property may exhibit a temperature dependence, in which case the applicator or other structure near the applicator heats the liquid hot melt adhesive immediately before application. The property may exhibit a pressure dependence, in which case the applicator or other structure near the application applies pressure and/or shear forces to the liquid hot melt adhesive shortly before dispensing. Alternatively, a property that is both temperature and pressure dependent may be modified.

### CROSS REFERENCE TO RELATED APPLICATIONS

This application claims the benefit of U.S. Provisional Application No. 60/562,043, filed Apr. 14, 2004, which is hereby incorporated by reference herein in its entirety.

### FIELD OF THE INVENTION

This invention relates generally to applicators and, more particularly, to applicators for dispensing liquid hot melt adhesive.

### BACKGROUND OF THE INVENTION

Applicators are routinely used to dispense viscous liquids, such as hot melt adhesives, sealants and other thermoplastics, in the manufacture of products and in product packaging. Conventional applicators incorporate dispensing guns or modules that regulate the discharge of the viscous liquid by operation of a valve assembly. Generally, such valve assemblies feature a valve seat positioned in a fluid path and a valve element that is movable for selectively contacting the valve seat to define opened and closed conditions and controlling the flow of liquid through the fluid path to a dispensing orifice. Cyclic movement between the opened and closed positions provides intermittent flow discontinuities in the liquid flow that generate a pattern in the liquid dispensed onto a surface of a product or product package.

The properties of liquid hot melt adhesives differ significantly from the properties of traditional hot melt adhesives. Traditional hot melt adhesives are converted from a room-temperature solid or semi-solid to a flowable form in a heated melter and are subsequently pumped through a heated hose to a heated manifold and applicator or gun. In contrast, liquid hot melt adhesives are flowable and have a relatively low viscosity at ambient or room temperature and pressure. When activated by, for example, exposure to elevated temperatures and/or pressures, liquid hot melt adhesives form a material of relatively high viscosity and adhesive properties similar to traditional hot melt adhesives and solidify upon cooling. The activated and solidified material behaves like a traditional hot melt adhesive and possesses similar bonding characteristics. An exemplary liquid hot melt adhesive disclosed in U.S. patent application Publication No. 2004/0029980 consists of discrete particle components dispersed in a carrier fluid and is heat activated.

As mentioned above, liquid hot melt adhesives are sensitive to an external factor, such as an elevated temperature and/or pressure, present or applied during the application process or shortly before application, and are activated by the action of the external factor. Activated liquid hot melt adhesives have a relatively short operating life and, hence, cannot be simply stored for use but must be dispensed shortly after activation. The development of liquid hot melt adhesives has led to a need for dispensing systems capable of locally activating amounts of liquid hot melt adhesive pumped to the gun as a room temperature liquid.

It would therefore be desirable to provide applicators and dispensing systems equipped for transferring, activating, and dispensing liquid hot melt adhesives.

### SUMMARY OF THE INVENTION

In accordance with one embodiment of the invention, a dispensing system includes a delivery system configured to supply a stream of the liquid hot melt adhesive in a non-activated first state. In the non-activated first state, the liquid hot melt adhesive has a first viscosity that is relatively low. The dispensing system further includes an applicator receiving the stream of the liquid hot melt adhesive from the delivery system. The applicator is adapted to modify the activation-sensitive property of the liquid hot melt adhesive for converting the liquid hot melt adhesive in the stream to an activated second state. In the activated second state, the liquid hot melt adhesive has a second viscosity higher than the first viscosity and possesses adhesive properties similar to traditional hot melt adhesives. Coupled with the applicator is a nozzle receiving the stream of the activated liquid hot melt adhesive from the applicator. The nozzle includes a dispensing orifice from which the liquid hot melt adhesive is dispensed in the second state.

The dispensing systems and applicators of the invention are capable of receiving a flow of a liquid hot melt adhesive in an unprocessed or non-activated state and processing the liquid hot melt adhesive to change at least one activation-sensitive property of the liquid hot melt adhesive proximate the point of application. As a result, the liquid hot melt adhesive may be transferred from a bulk container in a flowable condition favorable for accomplishing transfer and then activated after pumping, and before use, at the applicator.

The above and other objects and advantages of the invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a diagrammatic view of an applicator and a dispensing system in accordance with an embodiment of the invention;

FIG. 2 is a diagrammatic view of an applicator and a dispensing system in accordance with another embodiment of the invention;

FIG. 3 is a diagrammatic view of an applicator and a dispensing system in accordance with another embodiment of the invention;

FIG. 4 is a disassembled view of a heat exchanger/mixer for use in the applicator of FIG. 3 in accordance with an embodiment of the invention;

FIG. 5 is a disassembled view of a heat exchanger/mixer for use in the applicator of FIG. 3 in accordance with an alternative embodiment of the invention;

FIG. 6 is a disassembled view of a heat exchanger/mixer for use in the applicator of FIG. 3 in accordance with an alternative embodiment of the invention;

FIG. 7 is a disassembled view of a heat exchanger/mixer for use in the applicator of FIG. 3 in accordance with an alternative embodiment of the invention; and

FIG. 8 is a diagrammatic view of an applicator and dispensing system in accordance with another embodiment of the invention.

### DETAILED DESCRIPTION OF THE PREFERRED EMBODIMENTS

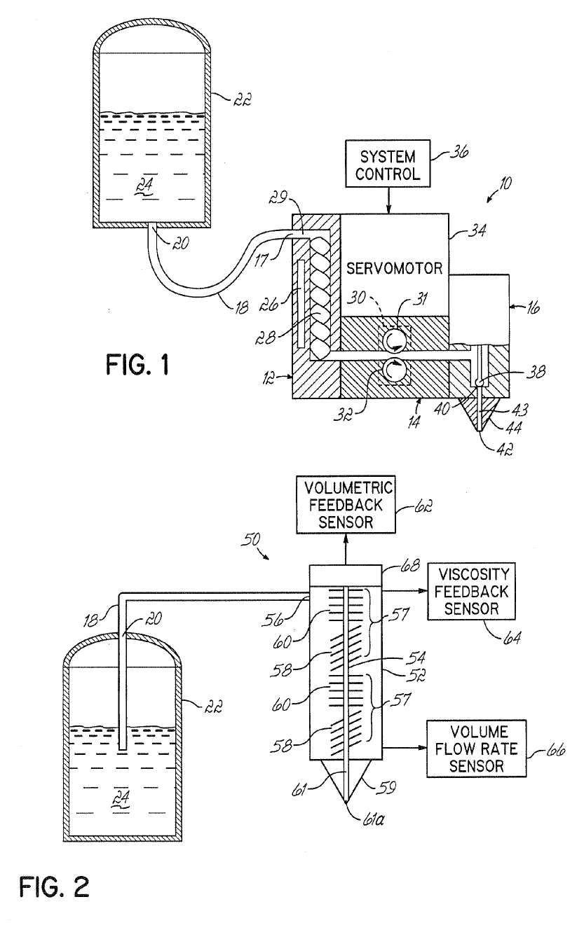

With reference to FIG. 1, an applicator 10 for intermittently dispensing viscous liquid hot melt adhesives includes a mixer/heat exchanger 12, a pumping module 14 and, optionally, at least one module or gun 16. The applicator 10 is mounted in a dispensing machine or system (not shown) in a known manner for dispensing liquid hot melt adhesive in discrete volumes, such as beads or dots, to provide an interrupted, non-continuous pattern on a moving substrate or as continuous beads or stripes. Multiple applicators 10 may be ganged together within the dispensing machine or system.

As used herein, liquid hot melt adhesive refers to any viscous liquid pumped from a bulk container in a first state and activated after pumping at, near, or proximate to an applicator by manipulating or modifying at least one activation-sensitive property of the liquid hot melt adhesive. The activation establishes a second state appropriate for adhesive bonding. For example, the changed property may be an increase in viscosity precipitated by a change in pressure and/or temperature or by shear forces after pumping. As a more specific example, the liquid hot melt adhesive may be activated by pressurization or by shear forces in the absence of heating or may be activated by heating alone. Although the exemplary mixer/heat exchangers are described herein as being adjoining or adjacent to the gun or nozzle, persons of ordinary skill in the art will recognize that the invention is not so limited. In particular, the mixer/heat exchangers described herein may be positioned at any location in the flow path from a bulk container to a gun or nozzle so long as the changed property does not adversely affect flow and/or materially affect dispensability.

A liquid inlet 17 of the mixer/heat exchanger 12 is coupled by a fluid hose 18 with a port 20 of a bulk container 22, such as a tote, a pressure vessel, or a drum, holding a relatively large volume of a liquid hot melt adhesive 24. The liquid inlet 17, fluid hose 18, and port 20 are equipped with hydraulic fittings for providing fluid-tight coupling for fluid flow. Liquid hot melt adhesive 24 is transferred at a relatively low pressure from the bulk container 22 through the fluid hose 18 to the liquid inlet 17 of the mixer/heat exchanger 12. In various embodiments of the invention and without limitation, the liquid hot melt adhesive 24 may be pumped by, for example, a diaphragm-type hydraulic pump from bulk container 22, may be drained from bulk container 22 by gravity, or the head space inside bulk container 22 may be pressurized as is characteristic of a pressure pot pump. The liquid hot melt adhesive 24 may be delivered to the mixer/heat exchanger 12 at an ambient temperature, which is frequently room temperature. An emptied bulk container 22 is simply replaced by a filled bulk container 22 to replenish the supply of liquid hot melt adhesive 24.

References herein to terms such as “vertical”, “horizontal”, etc. are made by way of example, and not by way of limitation, to establish a frame of reference. It is understood various other frames of reference may be employed because, as is well known, liquid dispensers may be oriented in substantially any orientation. Consequently, the directional words referenced herein should not be used to imply any particular absolute directions for an apparatus consistent with the invention.

With continued reference to FIG. 1, mixer/heat exchanger 12 includes a fluid heater 26 and a mixer 28 arranged for transferring heat to the liquid hot melt adhesive 24 and homogeneously mixing the components of the liquid hot melt adhesive 24. The heat transferred from the fluid heater 26 by a heat exchanger structure to liquid hot melt adhesive 24 flowing inside internal passageways 29 of mixer/heat exchanger 12 raises the temperature of the liquid hot melt adhesive 24 above the temperature measured at the liquid inlet 17 upon transfer from the bulk container 22. The elevation in temperature may be effective for transforming or changing the properties of the liquid hot melt adhesive 24. For example, the temperature increase may activate the liquid hot melt adhesive 24 so that it behaves like a conventional thermoplastic hot melt adhesive. The mixer 28 operates to homogeneously blend or mix any constituents or components, such as one or more active agents suspended in a carrier fluid, of the liquid hot melt adhesive 24.

Mixer 28 may be any conventional static mixer as recognized by a person of ordinary skill in the art that is compatible with installation in mixer/heat exchanger 12. Conventional static mixers, which have no moving parts, are devices having a series of internal baffles or elements, such as a series of alternating right- and left-hand helical elements oriented at right angles to one another. Portions of liquid hot melt adhesive 24 flowing through the mixer 28 are repeatedly divided into minor streams and recombined by operation of the baffles or elements, thus creating a substantially homogeneous mixture. The invention contemplates that the mixer 28 may be omitted if the liquid hot melt adhesive 24 has components that are not sensitive to separation, agglomeration, etc. and, hence, do not require thorough mixing to promote homogeneity.

Heating and mixing the liquid hot melt adhesive 24 proximate to the pumping module 14 and continuously delivering unprocessed liquid hot melt adhesive 24 to the applicator 10 permits processing and local activation only as required by the patterned bonding operation performed by the applicator 10. A separate melter and/or pump station and heated hoses are not required for dispensing the liquid hot melt adhesive 24. Hence, equipment cost is reduced and the design of the dispensing system is simplified. Processed liquid hot melt adhesive 24 may have a relatively brief work life and, therefore, may not be able to be stored for later use. Hence, applicator 10 facilitates local processing immediately of the liquid hot melt adhesive 24 before dispensing at the point of application. The liquid hot melt adhesive 24 is activated in the mixer/heat exchanger 12 upstream from the gun 16 and nozzle 44.

With continued reference to FIG. 1, the pumping module 14 may include a metering pump 30, illustrated as a gear pump having a counter-rotating set of gears 31, 32 driven by a servomotor 34 interfaced with a system control 36. The metering pump 30 elevates the pressure of the liquid hot melt adhesive 24 transferred from the mixer/heat exchanger 12 to the gun 16 and meters precise amounts of liquid hot melt adhesive 24. To that end, controlled rotation of the gears 31, 32 by servomotor 34 traps discrete metered amounts of liquid hot melt adhesive 24 in pockets or spaces defined between meshed gear teeth. As the gears 31, 32 rotate, the metered amounts of liquid hot melt adhesive 24 are supplied from the gears 31, 32 to an inlet of the gun 16 as necessary to fulfill pattern requirements. The pumping module 14 may also apply pressure to the liquid hot melt adhesive 24 for changing a property of adhesive 24.

One suitable pumping module 14 is the Prometer® VDK dispensing module commercially available from Nordson Corporation (Westlake, Ohio). Alternatively, the pumping module 14 may be a positive displacement metering unit with a single-acting or dual-acting piston displacement metering assembly capable of metering discrete amounts of the liquid hot melt adhesive 24.

Gun 16 may be any conventional hot melt dispenser, including but not limited to needle valve-type dispensers, capable of selectively actuating a valve element 38 relative to a sealing seat 40 for intermittently discharging liquid hot melt adhesive 24 and providing a positive cutoff of the flow of liquid hot melt adhesive 24. Gun 16 may be actuated electropneumatically and include a solenoid valve that supplies air pressure to an air cylinder for moving the valve element 38 away from a sealing seat 40, thereby allowing liquid hot melt adhesive 24 to flow into a passageway 43 defined in a removably-mounted nozzle 44. The passageway 43 terminates with a discharge orifice 42 from which the activated liquid hot melt adhesive 24 is dispensed. Alternatively, gun 16 may be electrically operated and include a coil that generates an electromagnetic field for moving an armature relative to a stationary pole, in which the valve element 38 is coupled for movement with the armature for moving the valve element 38 relative to the sealing seat 40. The removably-mounted nozzle 44 may be readily exchanged with other nozzles (not shown) for varying the configuration of discharge orifice 42 to dispense amounts, streams, dots or beads of viscous liquid characterized by a different size and/or a different shape.

Because of the precision metering of the liquid hot melt adhesive 24 provided by the pumping module 14, the requirement for a needle valve-type dispenser may be relaxed by omitting valve element 38 and sealing seat 40. Therefore, in an alternative embodiment of the invention, gun 16 is omitted and the removable nozzle 44 is mounted directly to the pumping module 14.

With reference to FIG. 2 in which like reference numerals refer to like features in FIG. 1 and in accordance with another embodiment of the present invention, an applicator 50 for dispensing liquid hot melt adhesive 24 includes a cylinder or barrel 52 and at least one screw 54 stationed within the barrel 52. The barrel 52, which may be heated, is a cylindrical housing in which the screw 54 rotates. The screw 54 is a helically flighted shaft that rotates within the barrel 52 to mechanically work and advance liquid hot melt adhesive 24 delivered via fluid hose 18 from bulk container 22 to a feed port 56 of the applicator 50. The space between the flight bounded by the screw 54 and the cylindrical bore of the barrel 52 defines at least one channel for fluid transport in the applicator 50.

Screw 54 is illustrated as a multi-stage screw having distinct serial stages 57 each with at least one dedicated feed section 58 and at least one mixing section 60. Each feed section 58 advances liquid hot melt adhesive 24 in the channel toward a nozzle 59 as screw 54 is rotated. Nozzle 59, which is removably mounted to the applicator 50, includes a passageway 61 receiving activated liquid hot melt adhesive 24 and a discharge orifice 61a terminating passageway 61 from which activated liquid hot melt adhesive 24 is dispensed from the applicator 50. Each mixing section 60 enhances distributive and/or dispersive mixing of the liquid hot melt adhesive 24. However, the invention is not so limited, as screw 54 may only have a single stage 57. Each stage 57 may also include distinct transition and metering sections. Each mixing section 60 may have a reduced helix angle and/or a special geometry so that liquid hot melt adhesive 24 has a lengthy dwell time and is advanced at a low rate along the length of the screw 54. The extended dwell time in the mixing section 60 may modify a property of the liquid hot melt adhesive 24 by the application of pressure and/or the introduction of heat.

The applicator 50 is equipped with sensors, such as a volumetric feedback sensor 62, a viscosity feedback sensor 64, and a volume flow rate sensor 66. Feedback from these sensors 62, 64, 66 may be used for controlling the amount of liquid hot melt adhesive 24 dispensed by increasing or decreasing the rotational speed of the screw 54. For example, an indication of the viscosity of the liquid hot melt adhesive 24 may be obtained by measuring the torque of a motor 68 driving the screw 54. Circuits capable of monitoring motor torque by sensing the back electromotive force (EMF) of the motor coils are well known and need not be described in detail herein.

The combination of mixing and heating in the applicator 50, in a manner similar to a conventional extruder, permits a rapid transformation of the liquid hot melt adhesive 24 from a non-activated state into an activated state in which adhesive 24 behaves like a thermoplastic hot melt material for dispensing onto a substrate. The pressure or shear forces applied to the liquid hot melt adhesive 24 inside the applicator 50 may also rupture any encapsulated component of adhesive 24 and, thereby, release that component for combining with other non-encapsulated components of adhesive 24. A separate heat exchanger is not required in this embodiment of the invention, as the mixing and heating is accomplished in a single unit presented by applicator 50.

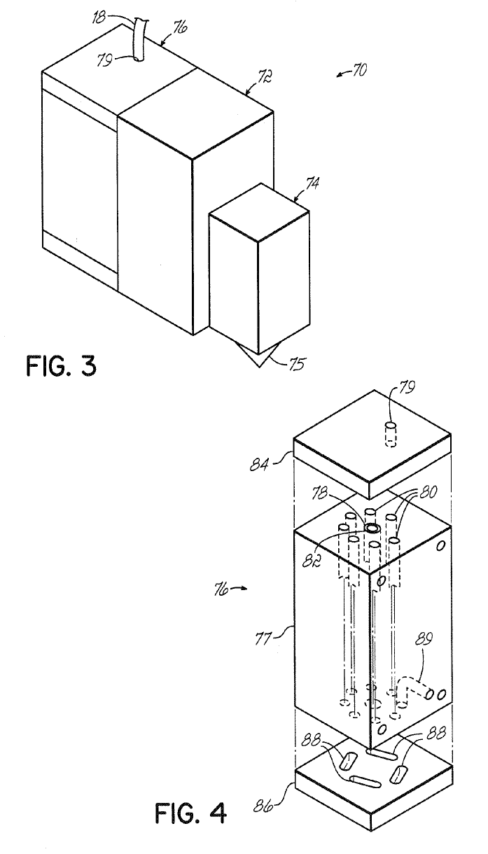

With reference to FIG. 3 in which like reference numerals refer to like features in FIGS. 1 and 2, an applicator 70 includes a distribution manifold 72, a module 74 coupled in fluid communication with the distribution manifold 72, and a mixer/heat exchanger 76 located in the fluid path between the bulk container 22 (FIG. 1) and the distribution manifold 72. Module 74 is similar or identical to gun 16 described above in the context of FIG. 1. A nozzle 75, which is removably mounted to the module 74, includes a passageway (not shown) receiving activated liquid hot melt adhesive 24 from the applicator 70 and having with a dispensing orifice from which activated liquid hot melt adhesive is dispensed. The distribution manifold 72, which is optional, distributes the liquid hot melt adhesive 24 to the module 74. The liquid hot melt adhesive 24 is activated in the mixer/heat exchanger 76 upstream from the module 74 and nozzle 75.

The mixer/heat exchanger 76 may assume any one of several configurations, as described below, and may optionally comprise a portion of the module 74. The invention contemplates that the mixer may be omitted from the mixer/heat exchanger 76 if the liquid hot melt adhesive 24 is constituted by components that are not sensitive to separation, agglomeration, etc. and, hence, do not require mixing to promote homogeneity. In an alternative embodiment, the mixer/heat exchanger 76 may be upstream from the applicator 70 and, hence, not a component of the applicator 70.

With reference to FIG. 4 and in accordance with one embodiment of the present invention, the mixer/heat exchanger 76 may be formed from a block 77 of a thermally-conductive material including a central bore 78 surrounded by a ring of fluid bores 80 each having an axial centerline aligned substantially parallel with an axial centerline of the central bore 78. Positioned in the central bore 78 is a heater element 82 connected with a system control (not shown). Heat is transferred through block 77 to liquid hot melt adhesive 24 resident and flowing inside fluid bores 80. Heat absorbed by the liquid hot melt adhesive 24 raises the temperature of the liquid hot melt adhesive 24 above the temperature measured at a liquid inlet 79 upon transfer through fluid hose 18 from the bulk container 22. As described herein, heating the liquid hot melt adhesive 24 may operate to modify its material properties. For example, the temperature increase may activate the liquid hot melt adhesive 24 so that it behaves like a conventional thermoplastic hot melt adhesive at, proximate to, or near the point of application.

An upper cap 84 and a lower cap 86 are each fastened to a corresponding one of the opposite upper and lower ends of the block 77. Lower cap 86 incorporates a plurality of blind slots 88 facing toward an open end of the fluid bores 80. Each of the blind slots 88 operates to transfer liquid hot melt adhesive 24 between adjacent fluid bores 80. The upper cap 84 incorporates a similar set of blind slots (not shown) similar to blind slots 88 facing toward the opposite open end of fluid bores 80. As a result, each successive volume of liquid hot melt adhesive 24 that enters the system of fluid bores 80 is constrained to flow serially through the entire set of fluid bores 80. This increases the residence time of the liquid hot melt adhesive 24 inside the block 77 and promotes heating to a suitable operating temperature. The serial flow through the fluid bores 80 and the abrupt changes in flow direction operate to homogenously mix the heated liquid hot melt adhesive 24. The liquid hot melt adhesive 24 is supplied from an outlet 89 coupled in fluid communication with the last in the series of fluid bores 80 defining the flow sequence to the distribution manifold 72.

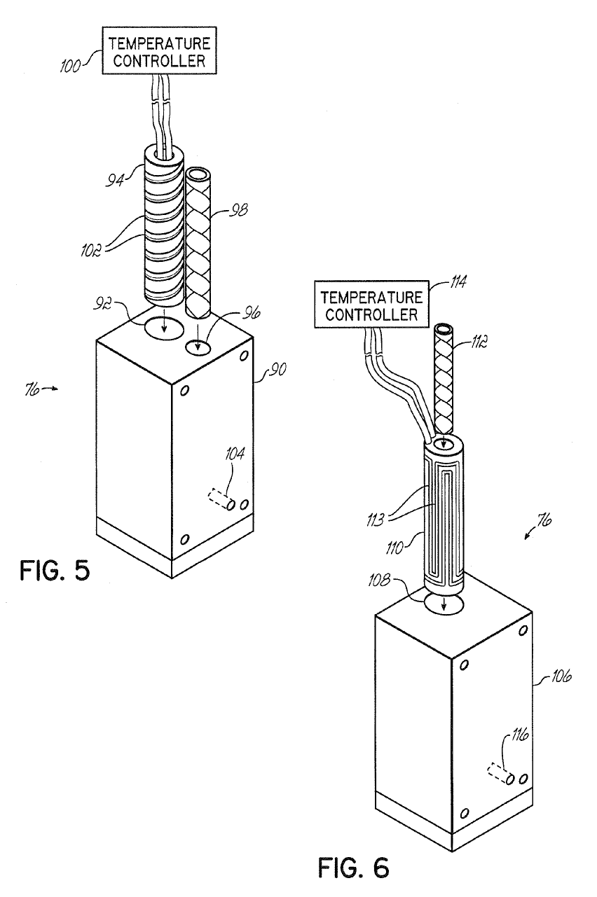

With reference to FIG. 5 and in accordance with another embodiment of the present invention, the mixer/heat exchanger 76 may be formed from a block 90 of a thermally-conductive material including a first bore 92 filled by a heater 94 and an adjacent, substantially parallel second bore 96 filled by a mixer 98. Heater 94 is a cartridge-style resistance heater coupled with a suitable temperature controller 100 that provides electrical energy for resistive conversion by the heater 94 into heat energy. Exemplary heaters 94 suitable for use in this embodiment of the mixer/heater exchanger 76 are commercially available under the FIREROD® trade name from Watlow Electric Manufacturing Company (St. Louis, Mo.).

Winding helically about the exterior of the heater 94 is an inscribed liquid channel 102 coupled at one end in fluid communication with liquid inlet 79 in an upper cap (not shown) similar to upper cap 84 (FIG. 4). The opposite end of the liquid channel 102 is coupled in fluid communication with an inlet of mixer 98. Heat transferred from the heater 94 to the liquid hot melt adhesive 24 flowing in the channel 102 is sufficient to elevate the temperature of liquid hot melt adhesive 24 to a desired operating temperature, as described elsewhere herein. A conventional temperature sensor (not shown), such as a resistance temperature detector (RTD), a thermistor or a thermocouple, provides a temperature feedback signal for use by the temperature controller 100 in regulating the power provided to the heater 94.

Mixer 98 operates to homogeneously blend or mix any constituents or components of the liquid hot melt adhesive 24. Mixer 98 may be similar or identical in design to mixer 28 (FIG. 1). Heat energy from the heater 94 is also transferred through the block 90 for further heating the liquid hot melt adhesive 24 during its residence time inside the mixer 98. A liquid outlet 104 supplies the mixed liquid hot melt adhesive 24 to the distribution manifold 72.

With reference to FIG. 6 and in accordance with yet another embodiment of the present invention, the mixer/heat exchanger 76 may be formed from a block 106 of a thermally conductive material including a bore 108 filled by a heater 110 wrapped about a mixer 112. The heater 110 and mixer 112 are arranged concentrically and preferably, have a coaxial arrangement. Heater 110 is a thick film cylindrical heater having a heating element 113 coupled with suitable temperature controller 114 that provides electrical energy for resistive conversion by the heating element 113 into heat energy. Liquid hot melt adhesive 24 is supplied to the mixer 112 from liquid inlet 79 in an upper cap (not shown) similar to upper cap 84 (FIG. 4).

Mixer 112 operates to homogeneously blend or mix any constituents or components of the liquid hot melt adhesive 24. The mixer 112 may be similar or identical in design and function to mixer 28 (FIG. 1) and mixer 94 (FIG. 5). Heat is transferred to liquid hot melt adhesive 24 flowing in the mixer 112. The dwell time of liquid hot melt adhesive 24 inside the mixer 112 is regulated, as is the power supplied from the temperature controller 114 to the heater 110, for elevating the temperature of liquid hot melt adhesive 24 to a suitable operating temperature, as described elsewhere herein. The heated liquid hot melt adhesive 24 is then routed from mixer 112 through a liquid outlet 116 from the mixer/heat exchanger 76 to the distribution manifold 72.

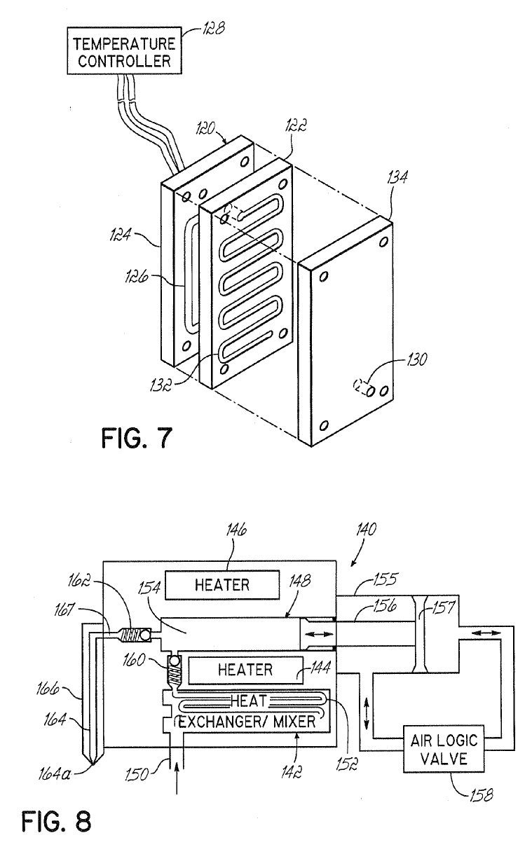

With reference to FIG. 7 and in accordance with yet another embodiment of the present invention, the mixer/heat exchanger 76 may be formed from a flat heater 120 coupled in thermal contact effective for efficient heat transfer with a separate flat heat exchanger 122. The flat heater 120 may be any flat, two-dimensional heater having the desired heating ability and appropriately sized to be compatible with the heater exchanger 122. Typically, the flat heater 120 has an area and a power density adequate to heat liquid hot melt adhesive 24 flowing in the heat exchanger 122 to a suitable process temperature and with a suitable temperature uniformity. The flat heater 120 is illustrated in FIG. 7 as a resistive heater consisting of a substrate 124 and a thick film heating element 126 formed of a material of relatively high resistivity that is electrically isolated from substrate 124. Due to its low thermal mass, thick film heating element 126 is highly responsive to variations in input power from a temperature controller 128. Exemplary flat heaters 120 suitable for use in accordance with this specific embodiment of the mixer/heater exchanger 76 are commercially available from Watlow Electric Manufacturing Company (St. Louis, Mo.). In an alternative embodiment, flat heater 120 may be a cartridge-style resistance heater, as described elsewhere herein, installed in a bore (not shown) formed in either heat exchanger 122 or a cover plate 134.

The temperature controller 128 supplies electrical energy to the heating element 126 that is resistively dissipated to produce thermal energy used for heating the liquid hot melt adhesive 24 flowing from liquid inlet 79 to a liquid outlet 130 through a convoluted channel 132 defined on a surface of heat exchanger 122 facing cover plate 134. A conventional temperature sensor (not shown), such as a resistance temperature detector (RTD), a thermistor or a thermocouple, embedded in the flat heater 120 senses the temperature of flat heater 120 and provides a feedback signal for use by the temperature controller 128 in regulating the power provided to the flat heater 120.

The channel 132, which receives liquid hot melt adhesive 24 supplied from the liquid inlet 79 (FIG. 3), winds back and forth across the surface of the heat exchanger 122 in a convoluted, folded, non-linear or serpentine path. This indirect path defined by channel 132 increases the effective path length and residence time for the liquid hot melt adhesive 24 inside the heat exchanger 122. The increased length of the path is achieved while minimizing the exterior dimensions of the heater exchanger 122. Cover plate 134 covers the channel 132 to close the fluid path for liquid hot melt adhesive 24 in the channel 132 of heat exchanger 122.

With reference to FIG. 8 and in accordance with another embodiment of the present invention, an applicator 140 includes a heat exchanger/mixer 142, a pair of heaters 144, 146, and a piston pump 148. Heaters 144, 146 may be any conventional heater design recognized by persons of ordinary skill in the art for heating applicator 140. In one specific embodiment, the heaters 144, 146 may be positive temperature coefficient (PTC) heating elements formed from a slug or ingot of a ceramic, such as barium titanate, that exhibits self-limiting temperature characteristics. Specifically, the electrical resistance of a PTC material increases dramatically with rising temperatures as voltage is applied across the ceramic ingot or slug so the heater temperature cannot exceed a characteristic maximum temperature. More specifically, a potential difference is established between metallic electrodes attached to opposite ends of the ceramic slug. The self-limiting temperature control eliminates the necessity of incorporating a temperature controller and a temperature sensing element into the system design. In an alternative embodiment of the invention, heaters 144, 146 may be conventional cartridge-style resistance heaters, as described elsewhere herein, installed in bores formed in the body of applicator 140.

The heat exchanger/mixer 142 incorporates a series of channels 152 through which liquid hot melt adhesive 24 is introduced from a liquid inlet 150 coupled with the bulk container 22 (FIG. 1) of liquid hot melt adhesive 24. The channels 152 are configured and arranged for promoting heat transfer from the heater 144 through the body of the applicator 140 to liquid hot melt adhesive 24 resident in the channels 152 by increasing the surface area of mutual contact. The liquid hot melt adhesive 24 may be mixed and blended, preferably homogeneously, by flow through the channels 152 due to flow constrictions and changes in flow direction of the channels 152. Alternatively, a separate mixer (not shown) similar or identical in design and function with mixer 28 (FIG. 1), mixer 94 (FIG. 5), and mixer 112 (FIG. 6) may be provided in the heat exchanger/mixer 142.

A piston pump 148 includes a fluid chamber 154 that receives liquid hot melt adhesive 24 in a heated and mixed state from the heat exchanger/mixer 142, an air cylinder 155, an air piston 157 disposed inside the air cylinder 155, and a plunger 156 extending from the air piston 157 into the fluid chamber 154. An air logic valve 158 regulates the air pressure supplied to the air cylinder 155 by alternatively filling and emptying air chambers defined inside the air cylinder 155 on opposite sides of the air piston 157 for reciprocating the air piston 157 relative to the air cylinder 155. Reciprocation of the air piston 157 moves the plunger 156 inside of the fluid chamber 154. Although the piston pump 148 is illustrated in FIG. 8 as bidirectional, the invention is not so limited. In particular, the piston pump 148 may be unidirectional and incorporate a return spring for shifting the air piston 157 to close the piston pump 148 and fill the fluid chamber 154. Other suitable actuation methods for the plunger 156 apparent to persons of ordinary skill in the art are contemplated by the invention.

A check valve 160 is situated in the passageway coupling the fluid chamber 154 with the heat exchanger/mixer 142. Another check valve 162 is situated in the passageway coupling the fluid chamber 154 with a passageway 164 in a nozzle 166, which is removably mounted to the applicator 140, by a discharge port 167 in fluid communication with the fluid chamber 154. Passageway 164 includes a discharge orifice 164a from which the activated liquid hot melt adhesive is dispensed. Check valves 160, 162 may be any suitable check valve that closes by fluid pressure to prevent return flow and that opens at a characteristic cracking pressure. In one embodiment, each of the check valves 160, 162 is characterized by a spring that biases a valve body or ball against a seat. The pressure inside of the fluid chamber 154 varies as the plunger 156 is moved therein, which regulates the opening and closing of check valves 160, 162. Exemplary check valves 160, 162 suitable for use in the invention are available commercially from The Lee Company (Westbrook, Conn.).

In use and with reference to FIG. 8, the plunger 156 is advanced within the fluid chamber 154 toward port 167 by concurrently introducing air pressure into air cylinder 155 above air piston 157 and exhausting air pressure from below air piston 157. During advancement of plunger 156, check valve 160 is configured or oriented to close so that additional liquid hot melt adhesive 24 cannot enter the fluid chamber 154. Check valve 162 is configured or oriented to open so that liquid hot melt adhesive 24 residing in the fluid chamber 154 is forced through the discharge port 167 into the passageway 164 in nozzle 166. The plunger 156 is advanced a set distance in a downstroke for forcing a volumetrically-metered amount of liquid hot melt adhesive 24 out of fluid chamber 154.

After dispensing a volume of liquid hot melt adhesive 24, plunger 156 is retracted inside fluid chamber 154 in a direction away from port 167 by introducing air pressure into air cylinder 155 below air piston 157 and exhausting air pressure from above air piston 157. During withdrawal of air piston 157 in this upstroke, check valve 160 is configured or oriented to open so that additional liquid hot melt adhesive 24 is drawn by suction into the fluid chamber 154 from heat exchanger/mixer 142 to replenish the enclosed reservoir and check valve 162 is configured or oriented to close. In this manner, the supply of processed liquid hot melt adhesive 24 in fluid chamber 154 is substantially refreshed with the occurrence of each dispensing cycle.

Applicator 140 is small and compact, which may be a benefit in adhesive applications such as for case sealing or bonding carton flaps. The liquid hot melt adhesive 24 is delivered to applicator 140 at a relatively-low pressure and, due to the action of the piston pump 148, has an increased pressurization only at the time of application. This may be important for pressure-sensitive liquid hot melt adhesives 24. Specifically, this may be important for liquid hot melt adhesives 24 that experience a property change upon the application of pressure above an established threshold. The liquid hot melt adhesive 24 is delivered to applicator 140 at a relatively-low temperature (e.g., room or ambient temperature), which may be important for liquid hot melt adhesives 24 that experience a property change, such as an increased viscosity, upon heating to an elevated temperature above an established threshold that would otherwise hinder pumping. Because heat is transferred from heater 144 to liquid hot melt adhesive 24 flowing in heat exchanger/mixer 142, the liquid hot melt adhesive 24 has an elevated temperature sufficient to activate a temperature-sensitive property of adhesive 24 only at or near the point of application. Hence, the liquid hot melt adhesive 24 is transformed from a non-activated state to an activated state upstream from nozzle 166.

Nozzle 166 is illustrated as a right-angle nozzle particularly suitable for applying liquid hot melt adhesive 24 to flaps on a carton or case. However, nozzle 166 may be a conventional nozzle with a linear passageway. Exemplary right-angle nozzles are the Saturn® right-angle nozzles for low profile packaging applications that are commercially available from Nordson Corporation (Westlake, Ohio).

While the invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. The invention in its broader aspects is therefore not limited to the specific details, representative methods, and illustrative examples shown and described. Accordingly, departures may be made from such details without departing from the spirit or scope of applicants' general inventive concept.

1. An apparatus for dispensing a liquid hot melt adhesive having at least one activation-sensitive property, comprising:
a delivery system configured to supply a stream of the liquid hot melt adhesive in a first state in which the liquid hot melt adhesive has a first viscosity;
an applicator receiving the stream of the liquid hot melt adhesive from said delivery system, said applicator adapted to modify the activation-sensitive property of the liquid hot melt adhesive for converting the liquid hot melt adhesive in the stream to a second state in which the liquid hot melt adhesive has a second viscosity higher than the first viscosity; and
a nozzle coupled with the applicator for receiving the stream of the liquid hot melt adhesive from said applicator, said nozzle including a dispensing orifice from which the liquid hot melt adhesive is dispensed.

2. The apparatus of claim 1 wherein said applicator includes a heater adapted to supply heat to the stream of the liquid hot melt adhesive to provide the second state.

3. The apparatus of claim 2 wherein said applicator further includes a heat exchanger heated by said heating element, said heat exchanger including at least one passageway through which the liquid hot melt adhesive flows while absorbing heat supplied by said heating element.

4. The apparatus of claim 3 wherein said applicator includes a mixer configured to blend the liquid hot melt adhesive before the liquid hot melt adhesive is converted to the second state.

5. The apparatus of claim 4 wherein said mixer and said heating element are concentrically arranged inside said heat exchanger.

6. The apparatus of claim 1 wherein said applicator is adapted to increase the pressure of the liquid hot melt adhesive to provide the second state.

7. The apparatus of claim 6 wherein said applicator includes a barrel and a rotatable screw disposed within said barrel to define a channel, said screw operative for mechanically working the liquid hot melt adhesive and thereby increasing the pressure of the liquid hot melt adhesive inside said channel to provide the second state.

8. The apparatus of claim 7 wherein said applicator further includes at least one sensor for monitoring the operation of said applicator.

9. The apparatus of claim 7 wherein said applicator includes a heater supplying heat to the stream of the liquid hot melt adhesive to cooperate with the pressure increase for providing the second state.

10. The apparatus of claim 1 wherein said applicator includes a mixer configured to blend the liquid hot melt adhesive supplied from said delivery system.

11. A method of dispensing a liquid hot melt adhesive, comprising:
supplying the liquid hot melt adhesive having at least one activation-sensitive property to an applicator in a first state in which the liquid hot melt adhesive has a first viscosity;
modifying the activation-sensitive property of the liquid hot melt adhesive for activating the liquid hot melt adhesive to provide a second state in which the liquid hot melt adhesive has a second viscosity higher than the first viscosity; and
dispensing the liquid hot melt adhesive in the second state onto a substrate.

12. The method of claim 11 wherein the activation-sensitive property is temperature dependent, and modifying the activation-sensitive property further comprises:
heating the liquid hot melt adhesive, before dispensing, to an elevated temperature higher than a supply temperature.

13. The method of claim 11 wherein the activation-sensitive property is pressure dependent, and modifying the activation-sensitive property further comprises:
exposing the liquid hot melt adhesive, before dispensing, to an elevated pressure higher than a supply pressure.

14. The method of claim 11 further comprising:
blending multiple components of the liquid hot melt adhesive to provide a mixture before dispensing.

15. The method of claim 11 wherein the liquid hot melt adhesive is supplied to the applicator at ambient temperature.

16. The method of claim 11 wherein the activation-sensitive property is modified by the applicator.
